(19) United States
(12) Patent Application Publication
(10) Pub. No.: US 2021/0145367 A1
(43) Pub. Date: May 20, 2021

(54) SYSTEMS, DEVICES, AND METHODS FOR DETERMINING INJURY RISK AND ATHLETIC READINESS

(71) Applicant: SPARTA SOFTWARE CORPORATION, Menlo Park, CA (US)

(72) Inventor: Phillip Patrick Wagner, Menlo Park, CA (US)

(21) Appl. No.: 17/107,057

(22) Filed: Nov. 30, 2020

Related U.S. Application Data
(63) Continuation of application No. PCT/US19/35361, filed on Jun. 4, 2019.
(60) Provisional application No. 62/680,783, filed on Jun. 5, 2018.

(51) Int. Cl. A61B 5/00; A61B 5/103; G16H 10/60; G16H 50/30; G16H 40/67; G06F 16/28

(52) U.S. Cl. CPC A61B 5/7275; A61B 5/1036; A61B 5/6892; A61B 5/742; A61B 2503/10; G16H 50/30; G16H 40/67; G06F 16/285; G16H 10/60

(57) ABSTRACT

Systems, devices and methods are provided for determining injury risk and athletic readiness based on athletic movement data. Generally, a sensor device, such as a force plate, is provided for sensing certain characteristics of an athletic movement. A local computing device coupled to the sensor device can be configured to receive sensor data indicative of the characteristics of the athletic movement, process and extract information from the sensor data, and transmit the processed sensor data to a remote server system. The remote server system can be configured to store, aggregate and update the processed sensor data in a database, and can also generate one or more normalized scores correlating to the athletic movement. The normalized scores can indicate to a user a susceptibility to injury and/or a readiness towards return to play.

### CROSS-REFERENCE TO RELATED APPLICATIONS

The subject application is a continuation of PCT Patent Application No. PCT/US19/35361, filed Jun. 4, 2019, which claims priority to U.S. Provisional Patent Application No. 62/680,783, filed on Jun. 5, 2018, both of which are incorporated by reference herein in their entirety for all purposes.

### FIELD

The subject matter described herein relates generally to systems, devices, and methods for determining injury risk and athletic readiness based at least in part on athletic movement data. In particular, sensor data for a user performing one or more athletic movements is captured by one or more sensors, analyzed, and transformed into one or more scores using a population database, or a subset thereof.

### BACKGROUND

Advances in kinesiology and sensor technology have enabled researchers to capture and study vast amounts of data regarding the human body during movement, and in particular, during athletic movement. Some researchers, for example, have utilized electromyography (“EMG”) for detecting and recording electrical activity produced by skeletal muscles during a golf swing. Other researchers have employed near-infrared spectroscopy (“NIRS”) to measure and monitor oxygenation in muscle and other tissues during cycling exercises. Still other researchers have tried affixing inertial sensors, or inertial measurement units (“IMUs”), to athletes during speed and agility exercises to quantify certain biomechanical metrics.

Despite an abundance and diversity of data, however, a significant challenge remains in translating the information gathered in these studies and others into actionable results. For example, every year the National Football League hosts the Combine, an event in which athletes are run through a battery of technical tests that purport to predict an athlete's likelihood of success as a football player. Athletes and agents spend significant amounts of money to prepare for the Combine, which utilizes high-technology sensors and other devices. Yet, a five-year study of Combine results for 306 football players concluded that there was no consistent statistical relationship between the Combine tests and professional football performance. In another study of basketball players in 2012, researchers were able to show a link between better performance and certain physical characteristics, such as a stiffer torso or a longer standing long jump. However, the study was unable to identify any meaningful patterns with respect to an athlete's injury risks and readiness.

Thus, needs exist for systems, devices and methods for objectively determining injury risk and athletic readiness.

### SUMMARY

Provided herein are example embodiments of systems, devices and methods for determining injury risk and athletic readiness. Generally, a local computing device is provided, where the local computing device is communicatively coupled to one or more sensors that are adapted to sense various characteristics of one or more athletic movements. These characteristics can include, for example, a plurality of ground reaction forces. The one or more sensors can be included within a single housing, such as that of a force plate. The local computing device, to which the one or more sensors are coupled, can also include, amongst other components, communications circuitry, one or more processors and a memory coupled to the one or more processors. The memory is configured to store instructions that, when executed by the one or more processors, cause the one or more processors to perform various method steps for determining injury risk and athletic readiness. For example, the local computing device can be configured to receive and process sensor data indicative of the various characteristics of the one or more athletic movements, and in turn, transmit the processed sensor data to a remote server system. The local computing device can also be configured to process, update, and transmit one or more user profiles to the remote server system. The user profiles can include data associated with dates, times, and frequency of the one or more athletic movement assessments for one or more associated users.

A remote server system is provided for receiving and storing the processed sensor data and user profile data, and can also be configured for transmitting back to the local computing device one or more scores, including, for example, injury risk scores, readiness scores, and other normalized scores correlating to the processed sensor data associated with the one or more athletic movements. The risk injury score can be based at least in part on one or more differential values of the processed sensor data. The readiness score can be based at least in part on the risk injury score and a frequency of athletic movement assessments. The scores can be T-scores, which can be normalized by various factors, such as by body weight, by gender, by preferred sport, by preferred position within a sport, or by injury risk. In addition, the remote server system can include, or be communicatively coupled to, a database comprising stored processed sensor data indicative of characteristics of various athletic movements for a population of athletes and user profiles. In this regard, and as described in further detail below, the injury risk scores, readiness scores, and normalized scores can provide a variety of objective and actionable data to a coach, a manager, or an athlete, such as, for example, susceptibility to injury, progression towards return to play, suitability for a particular sport, and/or readiness to play the particular sport at a given time, to name a few. Readiness can include at least the athlete's availability and ability to play.

To ensure the integrity of the acquired and processed sensor data, several data validation methods are also provided. For example, in some embodiments, prior to the user performing an athletic movement, the measured weight of the user is compared to a stored reference weight. If a weight mismatch is detected, e.g., if the measured weight is inaccurate or the user has misidentified herself, then the user is instructed to weigh in again. In other embodiments, one or more predetermined weight thresholds are monitored during the athletic movement which can detect, for example, a user prematurely stepping off the force plate, or the user not landing on the force plate with sufficient force. In still other embodiments, a final data check is performed before the processed sensor data is transmitted to the remote server system, which can be used to detect a corrupt file. These and other data validation methods are described in U.S. Patent Application No. 62/528,866, which is incorporated by reference in its entirety for all purposes.

These embodiments and others described herein are improvements in the fields of computer-assisted biomechanics and, in particular, in the area of computer-based kinetic and kinematic analysis. Other systems, devices, methods, features and advantages of the subject matter described herein will be apparent to one with skill in the art upon examination of the following figures and detailed description. The various configurations of these devices are described by way of the embodiments which are only examples. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of one or more local computing devices each of which can be coupled to a sensor device, a network, and a remote server system including a database.

FIG. 2 is a block diagram of an example embodiment of a local computing device.

FIG. 3A is a block diagram of an example embodiment of a remote server system.

FIG. 3B is a graph depicting a digital signal acquired during an athletic movement.

FIGS. 4A and 4B are flow chart diagrams depicting example embodiment methods for assessing a user's injury risk score.

FIGS. 4C to 4F are pictorial diagrams depicting certain steps in the example embodiment methods of FIGS. 4A and 4B.

FIG. 5 is a flow chart diagram depicting example embodiment methods for assessing a user's readiness score.

FIG. 6 is a flow chart diagram depicting an example embodiment method for generating an injury risk score of a user, including data validation steps.

FIG. 7 is an example embodiment of graphical user interface for displaying various athletic movement data and associated scores.

FIG. 8 is an example embodiment of a graphical user interface for displaying athletic movement data of a user on different dates.

FIG. 9 is an example embodiment of a graphical user interface for displaying athletic movement data of a user.

FIG. 10 is an example embodiment of a graphical user interface for displaying summary of various athletic movement data and associated scores for a plurality of users.

FIG. 11 is an example embodiment of a graphical user interface for displaying a comparison of athletic movement data and associated scores for a plurality of users.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for determining injury risk and athletic readiness based at least in part on athletic movement data. Accordingly, many embodiments can include one or more sensor devices coupled to one or more local computing devices, wherein the one or more sensor devices are configured to measure various characteristics of an athletic movement performed by a user. In addition, many embodiments can include a remote server system which can include, or be communicatively coupled with, a database configured to store processed sensor data associated with various athletic movements for a population of users.

In some embodiments, for example, a force plate can be configured to measure a resultant sway velocity associated with a user standing in a balance pose on the force plate. The resultant sway velocity is transmitted to a remote server system, and, subsequently, one or more normalized scores correlating to the resultant sway velocity are displayed on the local computing device. In these embodiments, the normalized scores can reflect a user's static stability.

In other embodiments, a force plate can be configured to measure a peak force and time to stabilize within a predetermined percentage of a reference weight associated with a user jumping from a stationary position to a landing position on the force plate. The peak force and time to stabilize are transmitted to the remote server system and, subsequently, one or more normalized scores correlating to the peak force and time to stabilize are displayed on the local computing device. In these embodiments, the normalized scores can reflect a user's dynamic stability.

Additionally, the present disclosure also includes systems and methods for validating the data acquired by the one or more sensors, and can include, for example, a weight mismatch process, a weight deviation process, a peak force deviation process, a premature end condition monitoring process, and a final data check process, among others, each of which is described in further detail below. The embodiments disclosed herein can include local computing devices, each of which is in communication with a remote server system that is location-independent, i.e., cloud-based. Those of skill in the art will also appreciate that the embodiments disclosed herein can also include local computing devices, each of which is in communication with a remote server system that is located on the same premise and/or local area network as the one or more local computing devices. In these embodiments, the remote server systems which are located on the same premise and/or local area network as the one or more local computing devices can also be configured to synchronize a database containing processed sensor data associated with a population of athletes with a database residing on, or coupled with, a centralized remote server system that is location-independent, i.e., cloud-based.

Furthermore, for each and every embodiment of a method disclosed herein, systems and devices capable of performing each of those embodiments are covered within the scope of the present disclosure. For example, embodiments of sensor devices, local computing devices, and remote server systems are disclosed, and these devices and systems can each have one or more sensors, analog-to-digital converters, one or more processors, memory for storing instructions, displays, storage devices, communications circuitries (for wired and/or wireless communications), and/or power sources, that can perform any and all method steps, or facilitate the execution of any and all method steps.

The embodiments of the present disclosure provide for improvements over prior modes in the field of computer-based kinetic and kinematic analysis. These improvements can include, for example, optimization of computer resources, improved data accuracy and improved data integrity, to name only a few. In a number of embodiments, for example, instructions stored in the memory of a local computing device (e.g., software) can cause one or more processors of the local computing device to process and extract certain characteristics from sensor data associated with one or more athletic movements received from a sensor device (e.g., a force plate), and transmit the processed sensor data to a remote server system. Subsequently, the remote server system receives and stores the processed sensor data, and returns to the local computing device one or more normalized scores correlating to the athletic movement. The normalized scores can be T-scores, for example, and displayed on the local computing device in an easy-to-read format, e.g., vertical bar chart. The sensor data on the local computing device can be subsequently discarded. Thus, according to one aspect of the embodiments, memory and hard drive space are conserved at the local computing device because sensor data need not be permanently stored. Likewise, the remote server system need only store processed sensor data (i.e., extracted values), and is not required to process or store raw sensor data, thereby conserving memory, hard drive space and processing power. Thus, computer resources can be significantly conserved both at the local computing device as well as at the remote server system.

The disclosed embodiments also reflect computer-related improvements in data accuracy and data integrity. In some embodiments, for example, the remote server system includes, or is communicatively coupled with a database for storing processed sensor data correlating to a population of athletes. According to one aspect of the disclosed embodiments, the remote server system can be location-independent (i.e., cloud-based), and configured to aggregate processed sensor data from a plurality of local computing devices, which can be located in a plurality of geographically dispersed areas. The remote server system can also provide normalized scores to each local computing system based on the population data contained in the database. The normalized scores can also be can be normalized according to categories, for example, by gender, by body weight, by sport or by position within a sport. By continually aggregating and updating the population data contained within the database, the remote server system can be configured to provide customizable, dynamically generated and accurate scores to the user.

According to another aspect of the disclosed embodiments, improvements in data integrity are also provided through data validation processes during the acquisition of the sensor data. As described in further detail below, the data validation processes can include, for example, a weight mismatch process, a weight deviation process, a peak force deviation process, a premature end condition monitoring process, a weight validation process, a minimum force process, a minimum velocity process, a minimum acceleration process, and a final data check process, among others. Each of these processes, as well as others, are configured to ensure that the acquired sensor data is accurate prior to processing and receiving the processed sensor data by the remote server system. Other sensor data validation processes are described in U.S. Patent Application No. 62/528,866, which is incorporated by reference in its entirety for all purposes.

The improvements of the present disclosure are necessarily rooted in computer-based systems for determining injury risk and athletic readiness based on athletic movement data, and are directed to solving a technological challenge that might otherwise not exist but for the existence of computer-based kinetic and kinematic analyses. Additionally, many of the embodiments disclosed herein reflect an inventive concept in the particular arrangement and combination of the devices, components and method steps utilized for acquiring, validating and analyzing athletic movement data. Other features and advantages of the disclosed embodiments are further discussed below.

Before describing these aspects of the embodiments in detail, however, it is first desirable to describe examples of devices that can be present within, for example, a system for determining injury risk and athletic readiness based on athletic movement data, as well as examples of their operation, all of which can be used with the embodiments described herein.

### Example Embodiment of Systems for Determining Injury Risk and Athletic Readiness

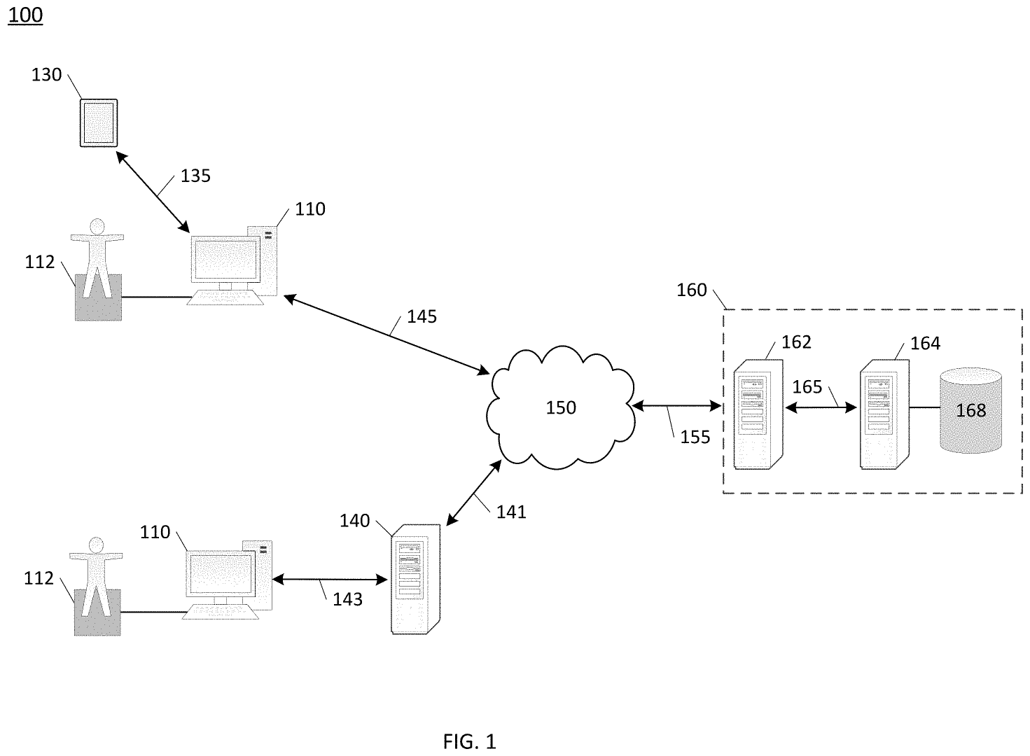

FIG. 1 is a conceptual diagram depicting an example embodiment of a system 100 for determining injury risk and athletic readiness based at least in part on athletic movement data, and which can be used with the embodiments of the present disclosure. System 100 includes a remote server system 160 configured to receive data from one or more computing devices 110, and which can comprise a front-end server 162 for interfacing with said computing devices 110, and a back-end server 164 that interfaces with both the front-end server 162 and database 168. Remote server system 160 can be a location-independent server system (e.g., cloud-based), which is accessible by a variety of computing devices 110 in geographically dispersed locations. Front-end server 162 can be in communication with back-end server 164 over a local area network, and can also communicate with computing devices 110 over communication path 155, which can include wired or wireless communications over network 150. In many of the embodiments disclosed herein, network 150 can be the Internet. In other embodiments, however, network 150 can also comprise one or more of an intranet, a wide area network, a local area network, a metropolitan area network, a virtual private network, a cellular network, or any other type of wired or wireless network. Furthermore, although front-end server 162 and back-end server 164 are depicted in FIG. 1 as two discrete devices, those of ordinary skill in the art will recognize that the functionalities and services of those devices can be implemented on a single centralized device or, in the alternative, can be distributed across multiple discrete devices in geographically dispersed locations. Likewise, those of skill in the art will recognize that the representations of servers in the embodiments disclosed herein, and as shown in FIG. 1, are intended to cover both physical servers and virtual servers (or “virtual machines”).

Referring still to FIG. 1, one or more local computing devices 110 are provided for receiving sensor data from sensor device 112, processing and extracting values from sensor data, and transmitting processed sensor data over network 150 to remote server system 160. Local computing device 110 can be a personal computer, laptop computer, wearable computer, desktop computer, workstation computer, or any other similar computing device, each of which can be communicatively coupled to a sensor device 112, which is configured to sense one or more athletic movements performed by a user. Sensor device 112 can be connected to local computing device 110 via a wired or wireless communication link. Additionally, as shown in FIG. 1, a mobile computing device 130, such as a tablet computer, laptop, smart phone, or wearable computing device, can also be communicatively coupled to local computing device 110 through a wired or wireless communication link. Mobile computing device 130 can be configured to send and receive data to and from sensor device 112 via computing device 110 through communication path 135. In other embodiments, however, mobile computing device 130 can be configured to communicate directly with sensor device 112 through Bluetooth, Bluetooth Low Energy, 802.11x, UHF, NFC or any other standard wireless communications protocol. In some of the embodiments, mobile computing device 130 is configured to operate according to a mobile operating system such as Android and/or iOS. Local computing device 110 can be configured to transmit and receive data over communication path 145 through network 150, which, as described earlier, can comprise the Internet, an intranet, a wide area network, a local area network, a metropolitan area network, a virtual private network, a cellular network, or any other type of wired or wireless network.

In some embodiments, a local server system 140 can reside on the same local area network as local computing device 110. Local server system 140 can receive and store processed sensor data from local computing device 110, and in turn, transmit locally stored injury risk scores, readiness scores, and other normalized scores to local computing device 110 over communications path 143. Local server system 140 can also synchronize a local database with the database 168 of the remote server system 160. In this regard, local server system 140 can serve as a proxy or intermediary between local computing device 110 and remote server system 160. In certain instances, this topology may be preferable, such as where heightened security is needed for local computing device 110 and/or the local area network on which local computing device 110 and local server system 140 reside. For example, the owner of local computing device 110 may not want to permit any or some of the processed sensor data collected through local computing device 110 to be transmitted to the remote server system 168, which may be shared by multiple tenants. In other instances, this topology may be preferable, for example where computing performance can be improved if sensor data can be processed locally at the local server system 140. Under those circumstances, local server system 140 can serve as a gateway, and conduct one-way synchronization or selective synchronization of the local database with database 168 of remote server system 160.

### Example Embodiment of Local Computing Device

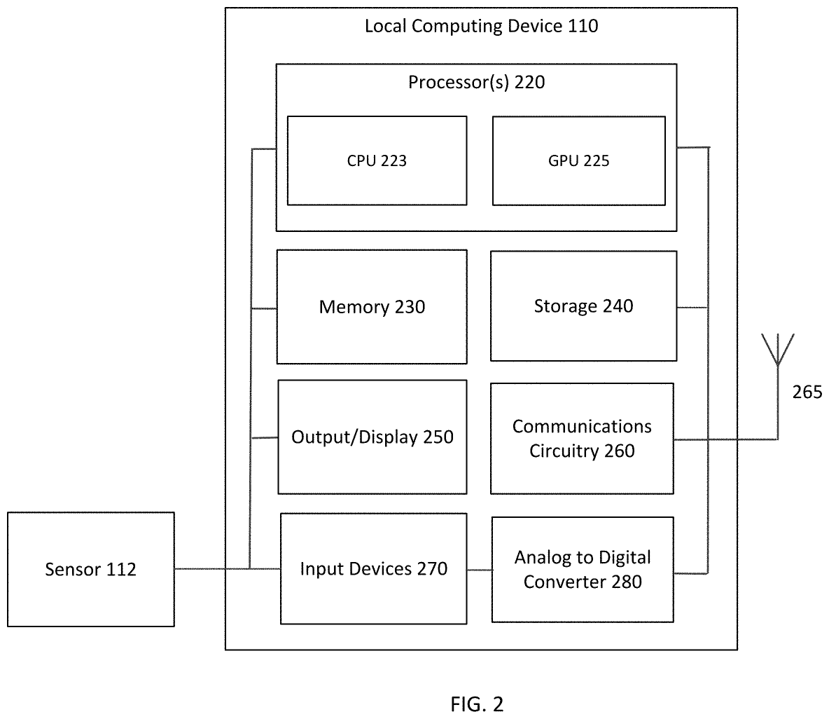

FIG. 2 is a block diagram depicting an example embodiment of local computing device 110. Local computing device 110 can include one or more processors 220, which can comprise, for example, one or more of a general-purpose central processing unit (“CPU”), a graphics processing unit (“GPU”), an application-specific integrated circuit (“ASIC”), a field programmable gate array (“FPGA”), an Application-specific Standard Products (“ASSPs”), Systems-on-a-Chip (“SOCs”), Programmable Logic Devices (“PLDs”), or other similar components. Processors 220 can comprise one or more processors, microprocessors, controllers, and/or microcontrollers, or a combination thereof, wherein each component can be a discrete chip or distributed amongst (and a portion of) a number of different chips, and collectively, can have the majority of the processing capability for acquiring, validating and analyzing athletic movement data. Local computing device 110 can also include memory 230, which can comprise non-transitory memory, RAM, Flash or other types of memory. Furthermore, local computing device 110 can include one or more mass storage devices 240, an output/display component 250, communications circuitry 260, which can include one or more wireless and/or wired network interfaces, an antenna 265 coupled to communications circuitry 260, an analog to digital converter component 280 configured to convert an analog signal received from a sensor device into a digital signal, and an input device component 270, which can include keyboards, mice, trackpads, touchpads, microphones and other user input devices, each of which can be communicatively coupled to local computing device 110 via a wired or wireless connection.

In many of the embodiments of the present disclosure, input devices component 270 can also include a sensor device 112, which can comprise one or more sensors configured to sense various characteristics of an athletic movement. In many embodiments, for example, sensor device 112 can comprise a force plate including one or more piezoelectric sensors within a single housing, wherein the one or more piezoelectric sensors are adapted to measure ground reaction forces while one or more athletic movements are performed by a user. In some embodiments, sensor device 112 can comprise a force plate including one or more strain gauge sensors within a single housing. In still other embodiments, sensor device 112 can include multiple types of sensors, in which data received from a first type of sensor can be used to corroborate the data received from a second type of sensor. For example, sensor device 112 can comprise a force plate including one or more piezoelectric sensors, as described earlier, and additionally, one or more accelerometers embedded within a portion of a user's footwear. Sensor data from the piezoelectric sensors and the accelerometers can be correlated, time synchronized and/or multiplexed by local computing device 110 to determine and corroborate various characteristics of the one or more athletic movements performed by the user. As understood by those of skill in the art, the aforementioned components are electrically and communicatively coupled in a manner to make one or more functional devices.

Referring still to FIG. 2, communications circuitry 260 of local computing device 110 can be configured to communicate directly with remote server system 160, or via local server system 140. In many of the embodiments disclosed herein, local computing device 110 is configured to receive sensor data generated by sensor device 112 in response to a user performing one or more athletic moves. The received sensor data can be processed and transmitted to remote server system 160 which, in turn, transmits one or more scores, such as injury risk scores, readiness scores, or one or more normalized scores correlating to the athletic moves performed by the user to the local computing device 110. In some embodiments, the received sensor data can be processed at the local server system 140 and synchronized with the remote server system 160. In many of the embodiments disclosed herein, the scores can be visually displayed through a user interface on local computing device 110. In some embodiments, for example, the scores can be T-scores that are visually displayed in an easy to read format, such as a vertical bar chart. In other embodiments, normalized scores can be depicted as a plotted line as a function of time. These graphical user interfaces, as well as other visual representations, can be generated by processors 220 in response to instructions, e.g., in the form of a locally installed application, which reside in memory 230 of local computing device 110.

As described earlier, local computing device 110 is represented in FIG. 2 as a personal computer, desktop computer, laptop computer or workstation. However, in some embodiments, the one or more local computing devices 110 can also include laptop computers, tablet computing devices, smartphones, personal digital assistants, wearable computing devices or other mobile computing devices.

### Example Embodiments of Remote Server System

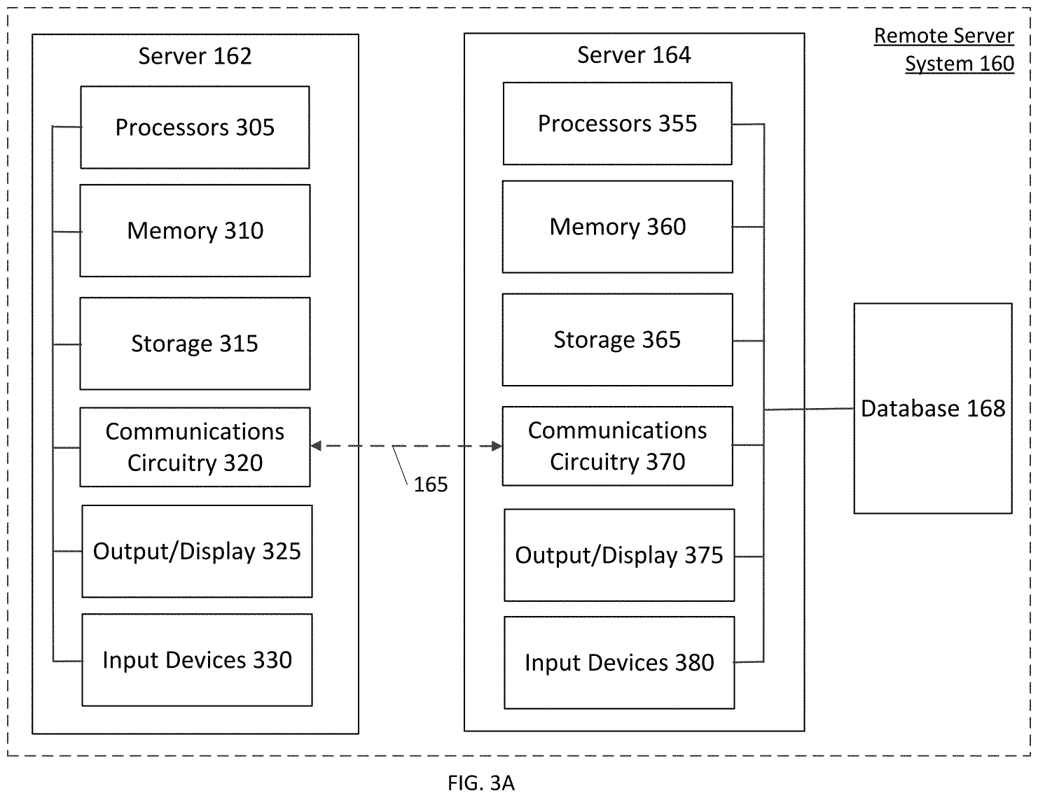

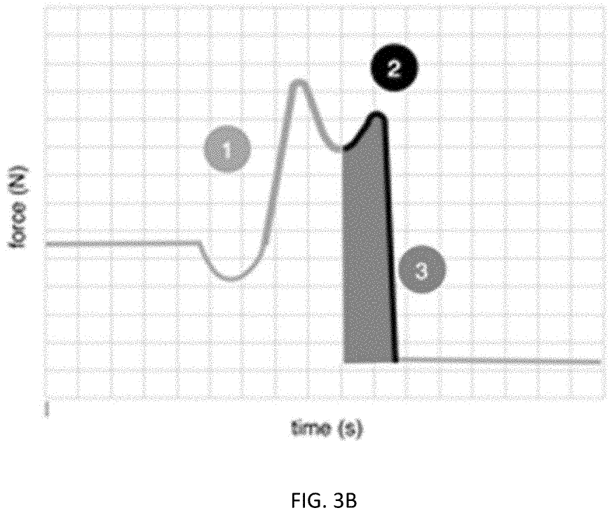

FIG. 3A is a block diagram depicting an example embodiment of remote server system 160 comprising one or more servers, and which can include a front-end server 162 and a back-end server 164. As shown in the diagram, servers 162, 164 can each include, respectively, an output/display component (325, 375), one or more processors (305, 355), memory (310, 360), including non-transitory memory, RAM, Flash or other types of memory, communications circuitry (320, 370), which can include both wireless and wired network interfaces, mass storage devices (315, 365), and input devices (330, 380), which can include keyboards, mice, trackpads, touchpads, microphones, and other user input devices. The one or more processors (305, 355) can include, for example, a general-purpose CPU, a GPU, an ASIC, an FPGA, ASSPs, SOCs, PLDs, and other similar components, and furthermore, can comprise one or more processors, microprocessors, controllers, and/or microcontrollers, each of which can be a discrete chip or distributed amongst (and a portion of) a number of different chips. As understood by one of skill in the art, these components are electrically and communicatively coupled in a manner to make a functional device.

In some embodiments, front-end server 162 can be configured such that communications circuitry 320 provides for a single network interface which allows front-end server 162 to communicate with the one or more local computing devices, as well as back-end server 164. In other embodiments, front-end server 162 can be configured such that communications circuitry 320 provides for two discrete network interfaces to provide for enhanced security, monitoring and traffic shaping and management. In addition, in most embodiments, front-end server 162 includes instructions stored in memory 310 that, when executed by the one or more processors 305, cause the one or more processors 305 to receive processed sensor data from one or more local computing devices, store processed sensor data to a database 168, and generate and transmit one or more scores associated with an athletic movement to a local computing device. In addition, the instructions stored in memory can further cause the one or more processors to perform one or more of the following routines: aggregate processed sensor data by various categories including by gender, by age, by body weight, by preferred sport and/or by position within a preferred sport; generate and store normalized scores associated with an athletic movement for one or more of the aforementioned categories; update scores based on newly received processed sensor data from the one or more local computing devices; and perform synchronization between database 168 and one or more databases residing on local server systems.

Referring still to FIG. 3A, server 164 can include database 168 for storing processed sensor data indicative of one or more characteristics of an athletic movement. In some embodiments, database 168 can reside on back-end server 164. In other embodiments, database 168 can be distributed or be part of a storage area network, for example, to which back-end server 164 is communicatively coupled. Back-end server 164 can also include communications circuitry 370 which can be configured to facilitate communications to and from front-end server 162. Similar to the configuration of front-end server 162, in many embodiments, communications circuitry 370 can include a single network interface, either wired or wireless; or, in other embodiments, communications circuitry 370 can include multiple network interfaces, either wired or wireless, to provide for enhanced security, monitoring and traffic shaping and management.

### Example Embodiments of Methods for Determining Injury Risk

Described herein are example embodiments of methods and systems for determining injury risk of a user based on athletic movement data. By way of background, FIG. 3B is a graph depicting portions of a digital signal extracted from sensor data acquired by, for example, local computing device 110 during an athletic movement performed by a user, such as when a user performs a vertical jump on a force plate coupled to local computing device 110. According to one aspect of the figure, the digital signal can be characterized by three measurements: (1) an average eccentric rate of force development; (2) an average relative concentric force divided by the weight of the user; and (3) a concentric relative impulse during the athletic movement. These sensor data are further described in U.S. patent application Ser. No. 14/050,735, which is incorporated by reference in its entirety for all purposes.

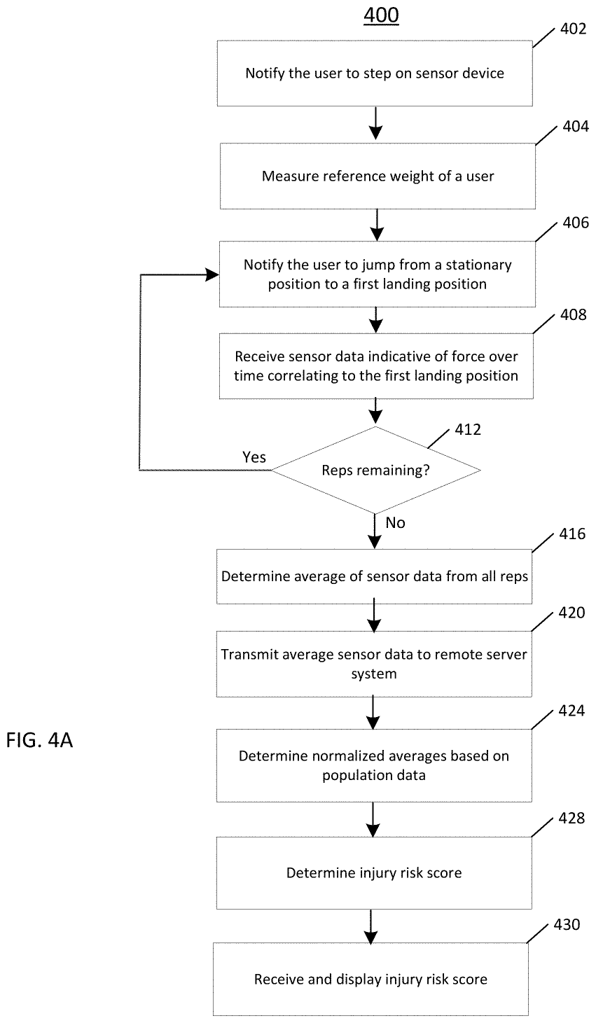

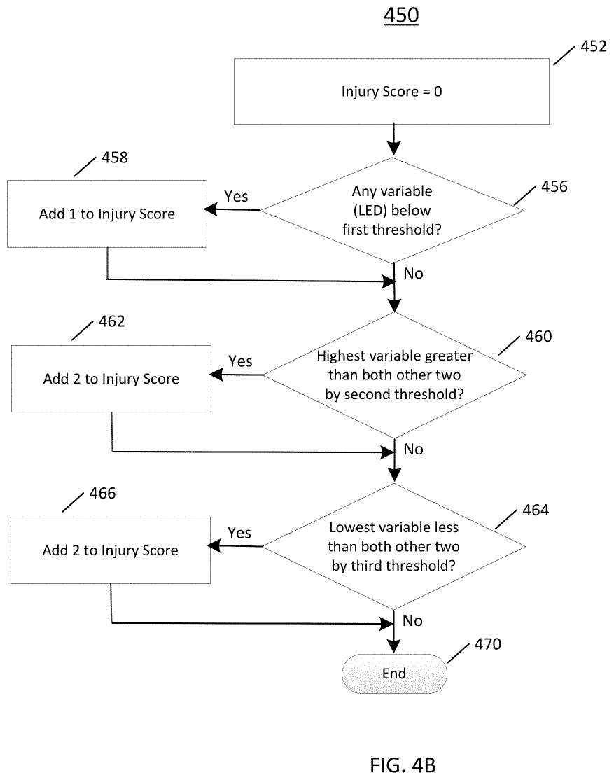

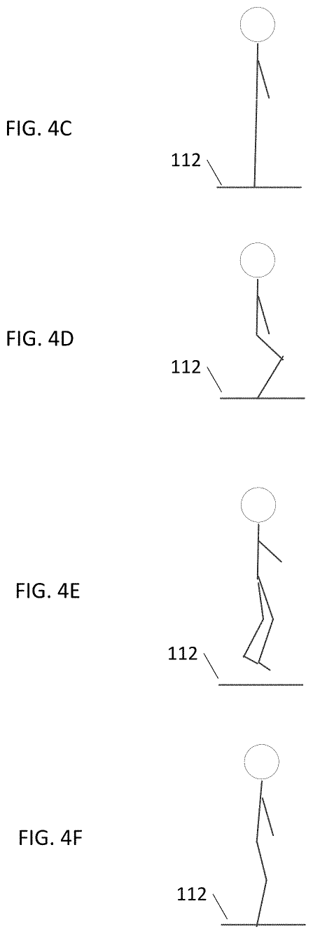

FIG. 4A is a flow diagram depicting an overview of an example embodiment of a method 400 for determining a user's injury risk score based on athletic movement data. Those of skill in the art will understand that the method steps disclosed herein can comprise instructions stored in memory of the local or mobile computing device, and that the instructions, when executed by the one or more processors of the local or mobile computing device, can cause the one or more processors to perform the steps disclosed herein.

As shown at the top of FIG. 4A, a user is instructed to step on to the sensor device at Step 402. At Step 404, the user's reference weight is measured. In some embodiments, the reference weight can be inputted manually by the user or another person. At Step 406, a visual or audio notification is outputted by the local or mobile computing device instructing the user to jump from a stationary position to a first landing position (as shown in FIGS. 4C to 4F).

At Step 408, while the user is in the first landing position, the local computing device receives sensor data from the sensor device, wherein the sensor data is indicative of the force generated by the user as a function of time. In some embodiments, the sensor data include at least the three aforementioned measurements: (1) an average eccentric rate of force development; (2) an average relative concentric force divided by the weight of the user; and (3) a concentric relative impulse during the athletic movement.

At Step 412, if it is determined that additional repetitions are required, the method returns to Step 406, and a visual or audio notification is outputted by the local or mobile computing device instructing the user to jump from the stationary position to another landing position. In some embodiments, a rest period can be interposed after Step 412, during which the user can rest and recover from the previous jump for a short period of time (e.g., 10 seconds) before being notified to perform the jump again at Step 406. In some other embodiments, six jumps are required. Those of skill in the art will appreciate that this number of repetitions is not meant to be exhaustive, and that other numbers of repetitions are fully within the scope of the present disclosure.

If it is determined that no repetitions are remaining then, at Step 416, the local or mobile computing device determines an average of sensor data measurements acquired during the repetitions. For example, an average eccentric rate of force development value can be calculated for the eccentric rate of force development measurements of all repetitions, an average relative concentric force (divided by the weight of the user) value can be calculated for the relative concentric force measurements of all repetitions, and an average concentric relative impulse value can be calculated for the concentric relative impulse measurements of all repetitions. At Step 420, the averaged sensor data measurements are transmitted to the remote server system. In some embodiments, an authentication step can be interposed after Step 416, prior to transmission, in order to ensure that the local or mobile computing device is authorized to transmit data to the remote server system. In some embodiments, the authentication step can be manual, such as requiring the user to input a password at the local or mobile computing device. In other embodiments, the authentication step can be automated through a public or private key exchange.

Referring still to FIG. 4A, at Step 424, one or more average sensor data measurements can be normalized based at least in part on the sensor data measurements for a predetermined population of athletes stored in a database residing on, or in communication with, the remote server system. In some embodiments, the predetermined population of athletes can comprise the entire population of athletes for which relevant data is stored in the database. In other embodiments, the predetermined population of athletes can comprise a subset of the entire population. Subsets of athletes can be categorized by gender, body weight range, age range, injury type, and position within a preferred sport. Those of skill in the art will appreciate that these examples are not meant to be exhaustive, and that other subsets of population data within the database are fully within the scope of the present disclosure. In addition, in many of the embodiments of the present disclosure, the determination of the normalized values can be performed by the one or more processors of the remote server system by either of the front-end server or the back-end server. In other embodiments, however, the determination of the normalized values can be performed elsewhere, such as, for example, by a local server system (as shown in FIG. 1), or by the local or mobile computing device itself.

According to one aspect of the embodiments of the present disclosure, the normalized values can be T-scores. T-scores enable a user to take a raw value (e.g., the processed sensor data) and transform it into a standardized score that allows the user to contextualize her assessment within a population of relevant athletes. A standardized score is typically determined by using the mean and standard deviation values from the relevant population data, as represented by the following equation:

$$z = \frac{x - μ}{σ}$$

where z is the standard score, x is the raw score (i.e., processed sensor data), μ is the mean of the relevant population, and σ is the standard deviation of the relevant population. The T-score is a standard z score shifted and scaled to have a mean of 50 and a standard deviation of 10. A standard z score can be converted to a T-score by the following equation:

T=(z×10)+50

In this regard, T-scores are both meaningful and easy to comprehend. Unlike other standardized measures (e.g., z-scores), T-scores are always positive and typically comprise whole integers. In addition, a T-score of over 50 is above average, a T-score of below 50 is below average, and each increment of 10 represents one standard deviation away from the mean value.

According to one aspect of some embodiments of the present disclosure, the normalized values for the averaged sensor data measurements—average eccentric rate of force development, average relative concentric force, and average concentric relative impulse—are referred to, respectively, as the Load value, the Explode value and the Drive value.

At Step 428, one or more injury risk scores can be determined based at least in part on the normalized values.

Referring to FIG. 4B, according to another aspect of some embodiments, a flow diagram depicting an overview of an example embodiment of a method 450 for determining a user's injury risk score is shown. Those of skill in the art will understand that the method steps disclosed herein can comprise instructions stored in memory of the local or mobile computing device, and that the instructions, when executed by the one or more processors of the local or mobile computing device, can cause the one or more processors to perform the steps disclosed herein. In some embodiments, an injury score can have a value range from 0 to 5, with a score of 5 indicating the greatest injury risk. At Step 452, the injury risk score can first be assigned a value of 0. At Step 456, if it is determined that any one of the normalized values (e.g., Load, Explode, Drive) is below a first predetermined threshold, a score of 1 is added to the injury score at Step 458. According to a first example, if the normalized values are 40, 55 and 85, and a first predetermined threshold is 45, a score of 1 is added to the injury score, which becomes 1 (or 0+1). In a second example, if the normalized values are 50, 55 and 65, and the first predetermined threshold is also 45, nothing is added to the injury score, which remains at 0.

Referring still to FIG. 4B, at Step 460, if it is determined that the highest value of the normalized values (e.g., Load, Explode, Drive) is higher than both of the other normalized values by at least a second predetermined threshold, a score of 2 is added to the injury score at Step 462. According to the first example above (where the normalized values are 40, 55 and 85), if the second threshold is 15, a score of 2 is added to the injury score, which becomes 3 (or 1+2). According the second example above (where the normalized values are 50, 55 and 65), nothing is added to the injury score, which remains at 0.

At Step 464, if it is determined that the lowest value of the normalized values (e.g., Load, Explode, Drive) is lower than both of the other normalized values by at least a third predetermined threshold, a score of 2 is added to the injury score at Step 466. In the first example above (where the normalized values are 40, 55 and 85), the third threshold is 15, a score of 2 is added to the injury score, which becomes 5 (or 3+2). In the second example above (where the normalized values are 50, 55 and 65), nothing is added to the injury score, which remains at 0. The method 450 then ends at Step 470. Those of skill in the art will appreciate that these examples are not meant to be exhaustive, and other sensor data variables and threshold values are fully within the scope of the present disclosure.

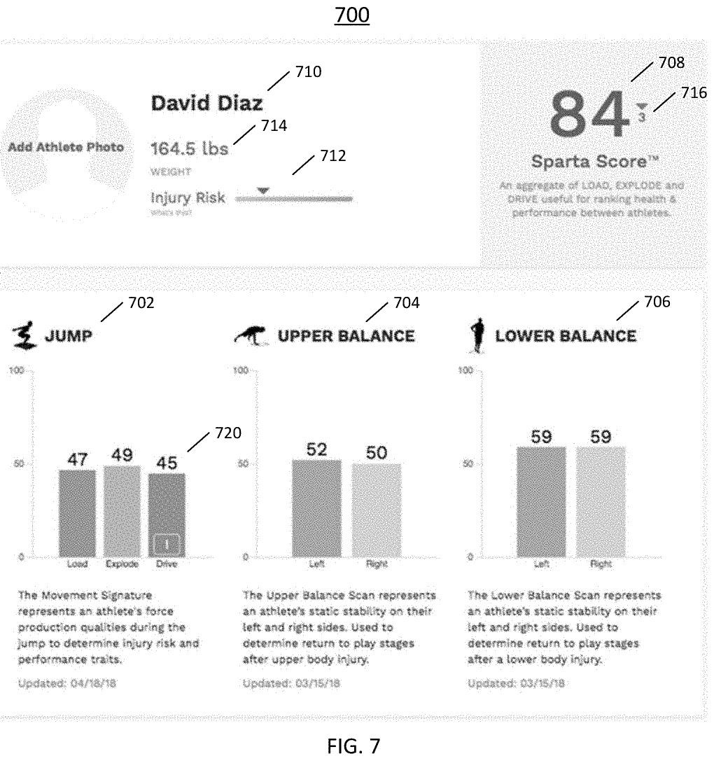

Referring back to FIG. 4A, at Step 430, the normalized scores are received by the local or mobile computing device and can be displayed in a graphical user interface, as can be seen in the embodiments depicted in FIGS. 7A, 7B and 7C.

In some embodiments, the data from the determination of injury risk are stored in the database. The data can include one or more of the weight of the user at the time of assessment, the averaged sensor value measurements, the normalized values, the injury score, and date and time of the assessment.

FIGS. 4C to 4F are pictorial diagrams depicting certain steps of methods 400, in which a user's injury risk is determined. FIG. 4C is a pictorial diagram showing the user stepping on and staying stationary on the force plate 112. FIG. 4D is a pictorial diagram showing the user in a countermovement position at the beginning of a vertical jump. FIG. 4E is a pictorial diagram showing the user in the middle of a vertical jump on the force plate 112, after leaving the stationary position on the force plate 112. FIG. 4F is a pictorial diagram showing the user as she lands on the force plate 112 on both legs. In some embodiments, the jump and land steps can be done with one leg, for example, with a leg previously injured, and repeated with the opposite leg. Although FIGS. 4C to 4F depict specific jumping and landing positions, these positions are meant to be illustrative and non-exclusive. Indeed, those of skill in the art will appreciate that other jumping and landing positions and techniques (e.g., landing with one foot, landing with two feet, landing on a designated portion of a foot, landing on a designated target on the force plate) are fully within the scope of the present disclosure.

### Example Embodiments of Methods for Determining Athletic Readiness

In some embodiments, a readiness score can indicate the availability and ability of one or more users, e.g., an athlete or an athletic team, to participate in a sport on the day of the assessment. In some embodiments, the readiness score is determined based at least in part on the user's injury risk score determined on the same day, and a frequency of assessments, or scans, the user has performed in the last predetermined length of time. According to one aspect of some embodiments, for example, a readiness score can be either 0 or 1. A readiness score of 0 may indicate that the user is not ready, or at 0 percent availability and ability to play. A readiness score of 1 may indicate that the user is ready, or at 100 percent availability and ability to play. A readiness for a group of users can be determined by averaging the readiness of each individual user on a particular day.

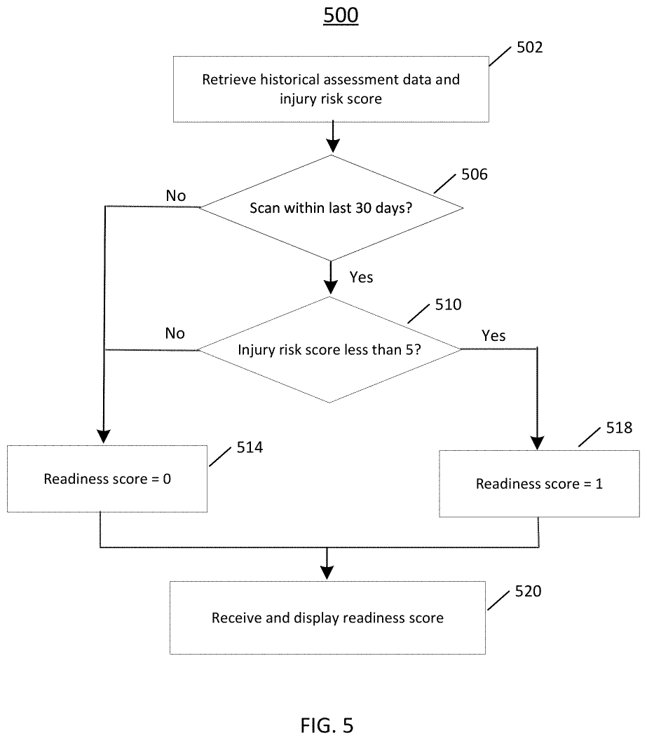

FIG. 5 is a flow diagram depicting an overview of an example embodiment of a method 500 for assessing a user's readiness score. Those of skill in the art will understand that the method steps disclosed herein can comprise instructions stored in memory of the local or mobile computing device, and that the instructions, when executed by the one or more processors of the local or mobile computing device, can cause the one or more processors to perform the steps disclosed herein. As shown at the top of FIG. 5, at Step 502, at least some of the user's historical assessment data are retrieved from a database residing on, or in communication with, the remote server system. In some embodiments, the data retrieved can comprise the history of frequency of assessments, or scans, of the user. At Step 506, if it is determined that the user has not performed any assessment within a predetermined length of time, the readiness score is assigned a value of 0 at Step 514. According to one aspect of some embodiments, the predetermined length of time can be 30 days. If it is determined that the user has performed an assessment within the predetermined length of time, a determination is made at Step 510 whether the user's injury risk score for the same day, is equal to a predetermined injury score threshold. According to one aspect of some embodiments, for example, the predetermined injury score threshold can be the maximum score, e.g., 5. If the user's injury risk score is equal to the score threshold, the readiness score is assigned a value of 0 at Step 514. If the user's injury risk score is less than the score threshold, the readiness score is assigned a value of 1 at Step 518.

At Step 520, the readiness score is received by the local or mobile computing device and can be displayed in a graphical user interface.

In some embodiments, a user's overall individual readiness score over time for a user can be determined by averaging historical readiness scores of the user.

In some embodiments, a group readiness score can be determined by averaging the readiness scores of all users in the group.

In some embodiments, to create a complete picture of a user's movement ability and her risk of injury, or how injury can affect the user, a balance test and a landing test can be performed for the user in conjunction with the vertical jump test described in FIGS. 4A-4B and 5 above. The balance test and the landing test are described in U.S. Patent Application No. 62/528,866, which is incorporated by reference in its entirety for all purposes.

In some embodiments, a combined test can be performed before or after an injury. In one aspect of some embodiments, after a user was injured, the order of the combined test can be: performing the balance test first, the landing test second, and the vertical jump test third.

### Example Embodiments of Methods for Athletic Injury Risk Data Validation

Example embodiments of methods for validating athletic movement data will now be described. Those of skill in the art will understand that the method steps disclosed herein can comprise instructions stored in memory of the local computing device, or in some alternative embodiments, in a mobile computing device or a remote server system, and that the instructions, when executed by the one or more processors, can cause the one or more processors to perform the steps disclosed herein.

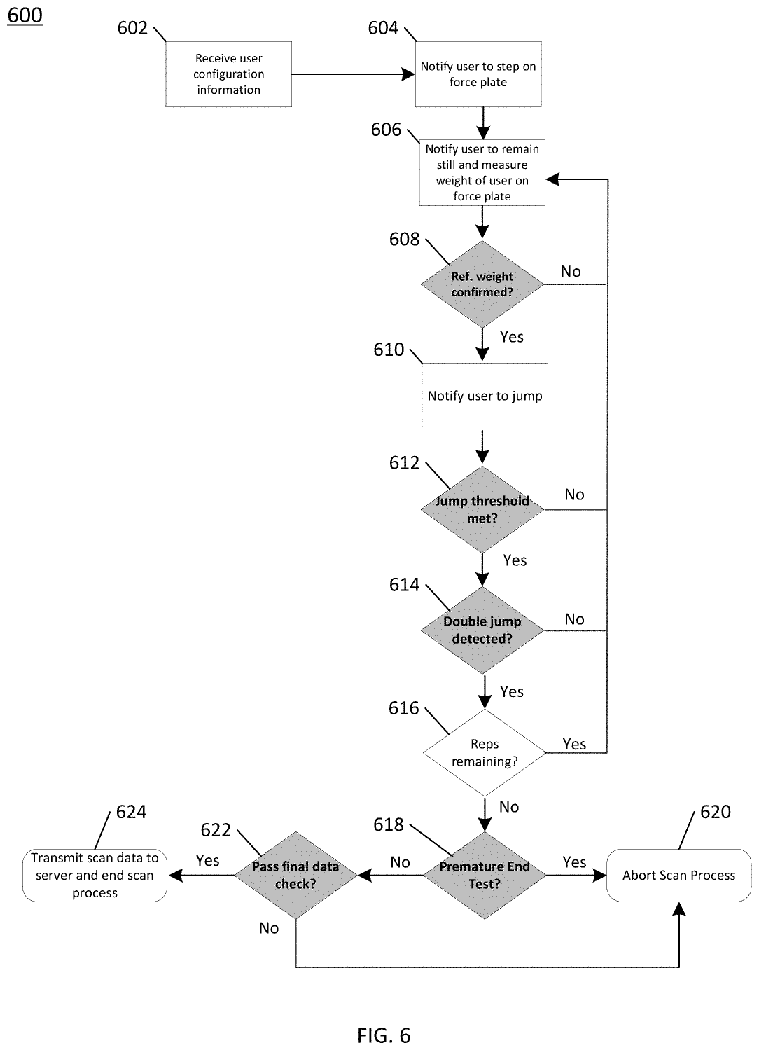

Referring first to FIG. 6, a flow diagram is provided, depicting an overview of an example embodiment of a method 600 for generating an injury risk score for a user, the method including one or more data validation steps (shown as shaded diamonds). At Step 602, user configuration information is received at a local computing system, or in some alternative embodiments, at a mobile computing device (e.g., tablet computer, smart phone, wearable computing device, etc.). The user configuration information can include any one or more of the following settings: a weight tolerance setting, a still criteria setting, injury risk threshold values setting, a readiness frequency value setting, an injury risk add-on values setting, an upward movement threshold setting, a jump error setting, a jump height threshold setting, a gender setting, a sport setting, and/or a position within a sport setting. At Step 604, a visual or audio notification is outputted by the local computing device instructing the user to step on the sensor device, i.e., the force plate. At Step 606, a visual or audio notification is outputted by the local computing device instructing the user to remain still while the sensor device measures the user's weight. At Step 608, the measured weight can be validated against a reference weight stored in memory for the particular user. For example, the validation can comprise checking if the measured weight is within a certain predetermined percentage of the stored reference weight for the user. In other embodiments, for example, the validation can comprise checking if the measured weight is within a certain predetermined percentage of the last weight measurement taken for the user. At Step 610, a visual or audio notification is outputted by the local or mobile computing device instructing the user to perform a vertical jump.

At Step 612, a determination can be made as to whether the user's jump has met a jump height threshold, which can include either or both of a minimum jump height and a maximum jump height. If the jump height threshold is not met, then the method returns to Step 606, and an audio or visual notification is outputted by the local or mobile computing device instructing the user to remain still while the sensor device measures the user's weight again. If the jump threshold is met, then at Step 614, another determination can be made as to whether a jump error, such as a double jump, has been detected. If a jump error has been detected, then the method returns to Step 606. If no jump error is detected, then at Step 916, a determination is made as to whether any repetitions remain. At Step 618, a determination is made whether the method has ended prematurely. A premature end may be determined, for example, if no repetitions are remaining, but there is an insufficient amount of data generated. A premature end may also be determined, for example, if the user steps off the sensor data and does not return before a timeout countdown has expired.

Referring still to FIG. 6, if no premature end is determined, then at Step 622, a final data check is performed. In some of the embodiments of the present disclosure, the final data check can include one or more steps taken to ensure that the file to be transmitted to the remote server system is not corrupted (e.g., CRC checksum). If the final data check is passed, then at Step 624, the data is transmitted to the remote server system.

### Example Embodiments of Graphical User Interfaces

Example embodiments of graphical user interfaces (“GUIs”) relating to the embodiments methods for injury risk determination and athletic readiness disclosed herein will now be described. Those of skill in the art will understand that the interfaces disclosed herein can comprise instructions stored in memory of the local computing device, or in some alternative embodiments, in a mobile computing device or a remote server system, and that the instructions, when executed by the one or more processors, can cause the one or more processors to create a visual output as described herein.

Referring first to FIG. 7, a GUI 700 is provided for displaying normalized and averaged sensor data measurements. The measurements may include athletic movement data 702 (e.g., normalized average eccentric rate of force development (Load), normalized average relative concentric force (Explode), and normalized average concentric relative impulse (Drive)—of a user. In exemplary GUI 700, athletic movement data 702 can comprise a Load value of 47, Explode value of 49, and Drive value of 45. Exemplary GUI 700 may also comprise other athletic movement data, including Upper Balance data 704 and Lower Balance data 706. Upper Balance data 704 can represent a user's static stability on the left and right sides of the upper body, and can reflect the user's condition with respect to return to play after an upper body injury. Lower Balance data 706 can represent a user's static stability on the left and right sides of the lower body, and can reflect the user's condition with respect to return to play after a lower body injury. In depicted embodiments 702, 704, and 706, the data is shown as vertical bars. Those of skill in the art will appreciate, however, that the normalized and averaged sensor data measurements can be displayed in other visual summary formats such as, for example, horizontal bars, pie charts, line plots, Harvey Balls, color coded indicators, etc., and are fully within the scope of the present disclosure. GUI 700 may also include the user's name or a unique identifier 710 (e.g., “David Diaz”), user weight 714 on the date of the assessment, and the injury risk score 712, as determined by the example embodiment methods described herein. In some embodiments (not shown), the data and time of the associated assessment may also be displayed. As shown in GUI 700, the injury risk score 712 can be displayed as a horizontal sliding scale which, in some embodiments, can include a color-coded indicator along the horizontal sliding scale. Those of skill in the art, however, will recognize that the injury risk score 720 can also be displayed in other visual summary formats, such as, for example, a vertical bar, a pie chart, a line plot, Harvey Balls, a color-coded indicator, etc., and are fully within the scope of the present disclosure.

Exemplary GUI 700 can also include a summary score 708, shown as the “Sparta Score,” which can represent a function of the user's injury risk and readiness as related to the user's Load, Explode and Drive values. A lower summary score 708 can indicate a higher injury risk and lower readiness. Conversely, a higher summary score 708 can indicate a lower injury risk and higher readiness. In this regard, the summary score 708 can be used to quickly compare multiple athletes. As shown, the summary score 708 can fall within a range between 0 and 100. In some embodiments, the summary score 708 can be determined by the following formula:

Sparta Score=average(((Average(L,E,D))+(min(L,E,D)))*0.75,100−((2*((max(L,E,D))−(min(L,E,D))))/7.5)),

where L is the Load value, E is the Explode value, and D is the Drive value.

In addition, according to some embodiments, an indicator 716 may also be displayed to indicate whether a user's summary score 708 has increased (upward-pointing green triangle) or decreased (downward-pointing red triangle) since the last assessment.

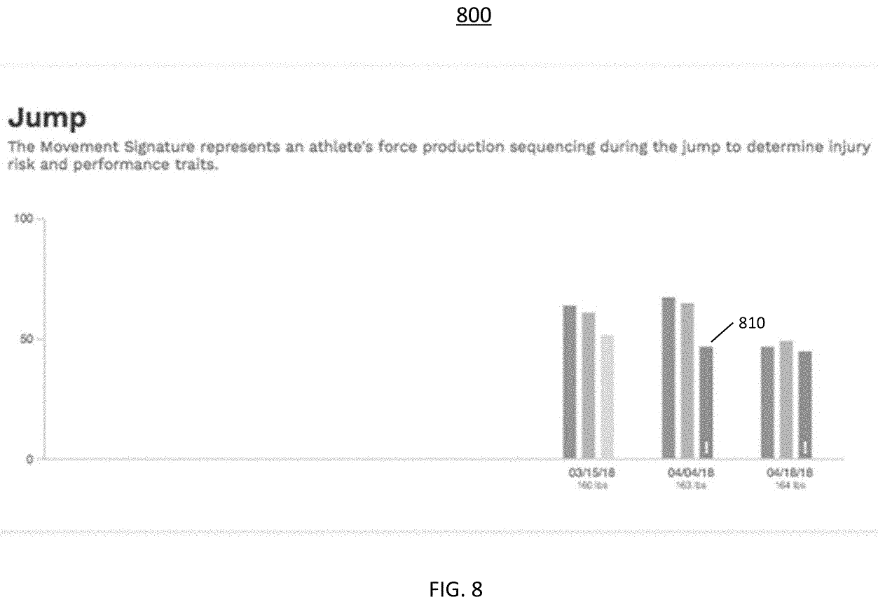

In some embodiments, the GUI 700 may display a particular data in a predetermined color different from the other data, or another special indicator, to indicate a source of a high injury risk score. In exemplary GUI 700, Drive value 720 is marked in a red color and includes an “!” mark to indicate that the user's Drive value is at or below a pre-determined threshold (e.g., 45). The predetermined threshold may be a source of a high injury risk score as described above in FIG. 4B. According to some embodiments, a predetermined color different from the other data, or a special indicator, may be displayed when a value is less than both of the other two measurements by a second predetermined threshold (e.g., 15), as described above in FIG. 4B, and as shown with exemplary value bar 810 in FIG. 8.

FIG. 8 is an exemplary GUI 800 displaying athletic movement data for a vertical jump (e.g., Load value, Explode value, and Drive value) of a user on three different dates. In some embodiments, a line plot (not shown) can also be displayed to show the injury risk on multiple dates, i.e., summarizing the progression of the individual over a predetermined time.

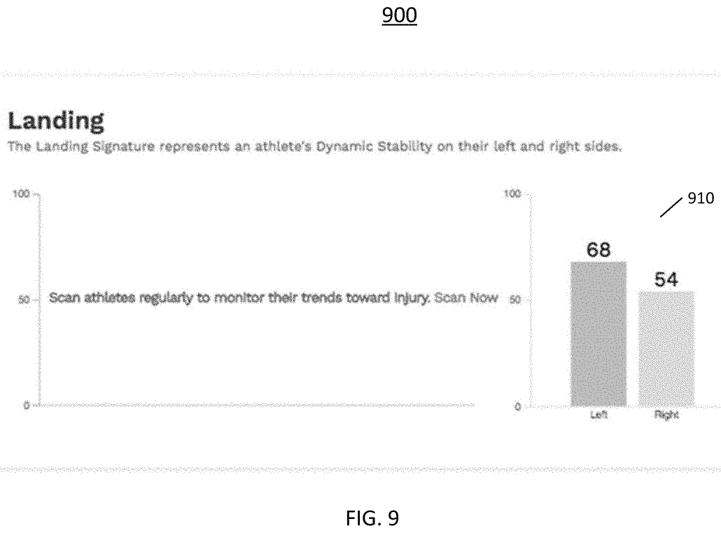

FIG. 9 is an exemplary GUI 900 displaying athletic movement data 910 for a landing movement of a user. According to one aspect of the embodiments, landing data 910 can represent the dynamic stability of the user's left and right side of a body after a jump onto a force plate.

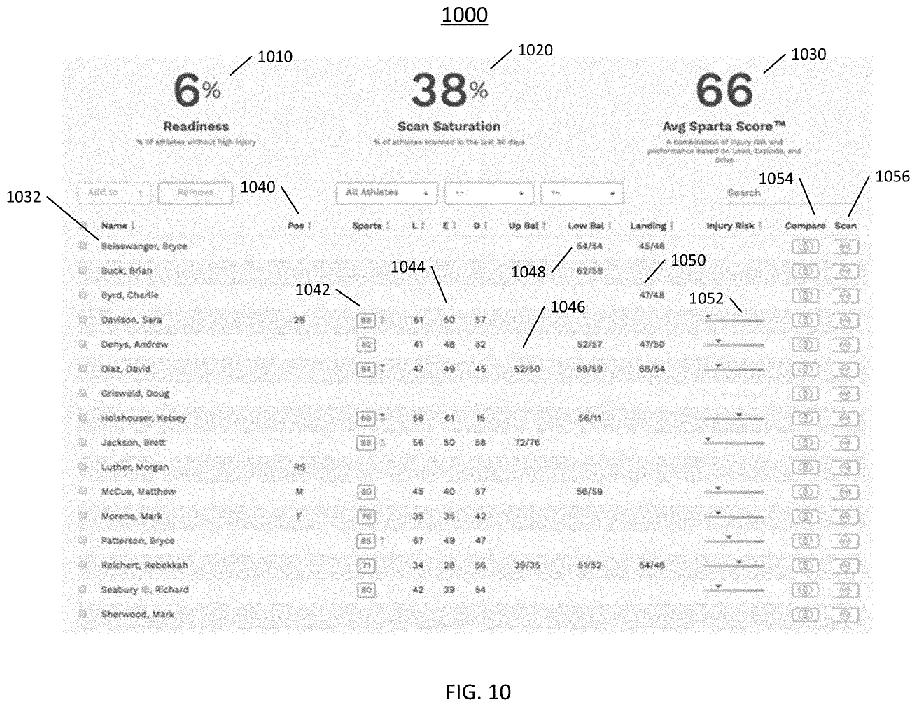

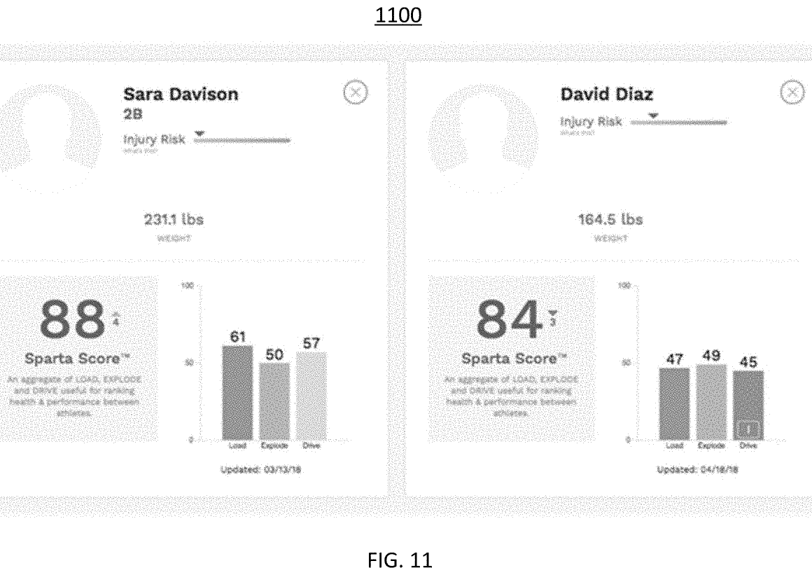

Turning to FIG. 10, an exemplary GUI 1000 is provided for displaying a summary of athletic movement data of a group of users such as, for example, multiple athletes on a team. GUI 1000 can include name 1032 of the user and the user's role or position 1040 for a particular sport. If athletic movement data has been acquired for a user (e.g., scanned), the athletic movement data for that particular user can be displayed, for example, Sparta Score 1042, Load, Explode and Drive values 1044, Upper Balance data 1046, Lower Balance data 1048, Landing data 1050, and Injury Risk score 1052. As can also be seen in GUI 1000, a “Compare” button 1054 may also be provided. According to one aspect of the embodiments, the “Compare” button 1054 can compare data, such as injury risk score, weight, Sparta score, etc., of the most recent scan of the one or more users. For example, FIG. 11 depicts an exemplary GUI 1100 comparing the most recent scan or measurement data of two users, “Sara Davison” and “David Diaz.” Referring back to FIG. 10, a “Scan” button 1056 may also be provided. According to one aspect of the embodiments, the “Scan” button 1056 can start a scanning process for the associated user. In some embodiments, an average injury risk score (not shown) can be displayed, consisting of the averaged injury risk scores of the users. In other embodiments, an athletic readiness score can also be displayed for each of the users, or as an average athletic readiness score of all of the users.

Also provided at the top of GUI 1000 are average data for the users in the group. For example, as shown in FIG. 10, the team has a 6% Readiness average (percentage of team users without high injury risk), 38% Scan Saturation (percentage of users scanned or tested in the last 30 days), and team average Sparta score of 66. Those of skill in the art will also appreciate that other central tendency values can be utilized, such as median, to summarize athletic movement data for a plurality of users.

It should also be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A computer-implemented method for assessing a user's injury risk, the method comprising:
measuring a reference weight of a user;
notifying the user to perform an athletic movement, wherein the athletic movement comprises a jump from a stationary position on a force plate to a landing position on the force plate;
receiving sensor data from the force plate during the athletic movement, wherein the sensor data comprises one or more force measurements over time;
determining one or more averages of the one or more force measurements;
transmitting the one or more averages to a remote server system;
normalizing the one or more averages based on a database residing on or in communication with the remote server system;
determining an injury risk score based on the one or more normalized averages; and
receiving from the remote server system and displaying on a local computing device the injury risk score.

2. The method of claim 1, wherein the one or more force measurements over time includes at least one of an eccentric rate of force development measurement, a relative concentric force measurement, and a concentric relative impulse measurement.

3. The method of claim 1, wherein the steps of notifying the user to jump and receiving a set of sensor data are repeated a plurality of times.

4. The method of claim 3, wherein the step of determining the one or more averages comprises averaging each of the one or more force measurements across the plurality of repetitions.

5. The method of claim 1, wherein the step of determining an injury risk score further comprising:
assigning a value of zero to the injury risk score;
adding one to the injury risk score if any of the one or more normalized averages is below a first threshold;
adding two to the injury risk score if a highest value of the one or more normalized averages is greater than the other normalized averages by a second threshold; and
adding two to the injury risk score if the lowest value of the one or more normalized averages is less than the other normalized averages by a third threshold.

6. The method of claim 5, wherein the first threshold is forty-five.

7. The method of claim 5, wherein the second threshold is fifteen.

8. The method of claim 5, wherein the third threshold is fifteen.

9. The method of claim 1, further comprising:
retrieving historical assessment data from a database;
determining a readiness score based at least on the retrieved historical assessment data, a predetermined length of time, and the injury risk score.

10. The method of claim 9, wherein the retrieved historical data include at least a frequency data.

11. The method of claim 10, wherein the frequency data include one or more historical dates and times associated with the user's historical injury scores.

12. The method of claim 11, further comprising assigning a value of zero to the readiness score if a date of the user's most recent injury score is greater than the predetermined length of time.

13. The method of claim 12, wherein the predetermined length of time is thirty days.

14. The method of claim 11, further comprising assigning a value of zero to the readiness score if the user's injury score is greater than a predetermined injury risk score threshold.

15. The method of claim 14, wherein the predetermined injury risk score threshold is a maximum injury score value.

16. The method of claim 15, wherein the maximum injury score value is five.

17. The method of claim 11, further comprising assigning a value of one to the readiness score if the user's last date of assessment of injury score is less than the predetermined length of time, and the user's injury score is less than the predetermined injury risk score threshold.

18. The method of claim 1, further comprising storing, by the remote server system, the injury risk score in the database.

19. The method of claim 1, further comprising storing, by the remote server system, date and time of the assessment in the database.

20. The method of claim 1, wherein normalizing the one or more averages includes utilizing a subset of the database, wherein the subset of the database comprises data categorized by at least one of gender, body weight range, age range, injury type, sport, or position within a sport.
